(19) United States
(12) Patent Application Publication
(10) Pub. No.: US 2017/0007132 A1
(43) Pub. Date: Jan. 12, 2017

(54) METHODS AND SYSTEMS FOR ASSESSING PERIPHERAL ARTERIAL FUNCTION

(71) Applicant: DREXEL UNIVERSITY, Philadelphia, PA (US)

(72) Inventors: Leonid Zubkov, Philadelphia, PA (US); Michael Neidrauer, Ardmore, PA (US); Joshua Samuels, Philadelphia, PA (US)

(73) Assignee: Drexel University, Philadelphia, PA (US)

(21) Appl. No.: 15/115,354

(22) PCT Filed: Feb. 25, 2015
(86) PCT No.: PCT/US15/17458

§ 371 Date: Jul. 29, 2016

Related U.S. Application Data
(60) Provisional application No. 61/946,826, filed on Mar. 2, 2014.

(51) Int. Cl. A61B 5/00; A61B 5/022; A61B 5/026

(52) U.S. Cl. CPC A61B 5/0075; A61B 5/0261; A61B 5/022

(57) ABSTRACT

One aspect of the invention provides a method for assessing peripheral arterial function in a subject. The method includes: conducting diffuse correlation spectroscopy on a local region of the subject; applying pressure to restrict blood flow to the local region for a period of time; conducting diffuse correlation spectroscopy on the local region while the pressure is applied; releasing the pressure; and conducting diffuse correlation spectroscopy on the local region after the pressure is released. Another aspect of the invention provides a system including: a diffuse correlation spectroscopy device and a pressure cuff.

### CROSS-REFERENCE TO RELATED APPLICATION

This application claims priority to U.S. Provisional Patent Application Ser. No. 61/946,826, filed Mar. 2, 2014. The entire content of this application is hereby incorporated by reference herein.

### BACKGROUND

PAD affects 10 million Americans and adds $4.4 billion to annual US healthcare costs. In addition to the direct costs, as the leading cause of amputation, peripheral arterial disease and its associated co-morbidities cost Americans upwards of $150 billion each year. Unfortunately, PAD is under-diagnosed and undertreated for the 16.5 million asymptomatic Americans. The narrowing or blocking of arteries can cause pain in the legs (called intermittent claudication), lead to strokes, or result in complete loss of circulation in limbs causing gangrene and loss of limb.

Cigarette smoking is the most important risk factor for development of PAD. Forty-four million American's smoke, which results in impaired arterial health, costing the United States over $139 billion ($96 billion in healthcare costs and $97 billion in lost productivity). It has been well documented that cigarette smoking, even passive (secondhand) tobacco exposure 20 years later, can lead to endothelial cell dysfunction, diagnosed as an impairment of the vessel's ability to expend during reactive hyperemia.

### SUMMARY OF THE INVENTION

One aspect of the invention provides a method for assessing peripheral arterial function in a subject. The method includes: conducting diffuse correlation spectroscopy on a local region of the subject; applying pressure to restrict blood flow to the local region for a period of time; conducting diffuse correlation spectroscopy on the local region while the pressure is applied; releasing the pressure; and conducting diffuse correlation spectroscopy on the local region after the pressure is released.

This aspect of the invention can have a variety of embodiments. The local region can be a ball of the subject's foot. The pressure can be applied by a blond pressure cuff. The pressure can be equal to or greater than the subject's systolic blood pressure. The pressure can be about 25 mm Hg greater than the subject's systolic blood pressure. The method can further include calculating a spike between blood flow while the pressure is applied and blood flow after the pressure is released. The method can further include calculating, a duration between release of the pressure and a peak of the spike.

The method can further include calculating a duration between release of the pressure and a return of blood flow to a pre-pressure level.

The period of time can be selected from the group consisting of: between about 1 minute and about 2 minutes, between about 2 minutes and about 3 minutes, between about 4 minutes and about 5 minutes, and greater than about 5 minutes.

The step of conducting diffuse correlation spectroscopy can include; applying light to a first location of the subject's skin; detecting photons resulting from interactions between the light and moving objects under the subject's skin; correlating, arrival times of the photons with light scattered intensity; and calculating a diffusion coefficient based on autocorrelation of the light scattered intensity.

The light applied to the subject's skin can be near-infrared light. The light applied to the subject's skin can have a wavelength between about 650 nm and about 1,000 nm. The light applied to the subject's skin can have a wavelength of about 785 nm. The light can be generated by a long-coherence laser. The laser can have a coherence of about 10 m. The light applied to the subject's skin can be conveyed to the subject's skin by a multimode optical fiber.

Light can be detected on a surface of the skin at a second location between about 1 mm and about 6 cm from the first location. The second location can be about 1.1 cm from the first location.

The correlating step can utilize a multi-tau autocorrelation algorithm.

The photons can be detected via, one or more single mode optical fibers. The one or more single mode optical fibers can each have a diameter of about 5 microns.

The steps of conducting diffuse correlation spectroscopy can further include generating a transistor-transistor logic (TTL) pulse each time a photon is detected.

The steps of conducting diffuse correlation spectroscopy can further include performing diffuse near-infrared spectroscopy (DNIRS) to determine the skin's optical scattering and absorption coefficients.

The steps of conducting diffuse correlation spectroscopy can further include solving the equation

$${g}_{1} ( ρ , τ ) = \frac{3 {μ}_{s}^{′}}{4 π} ( \frac{{e}^{- {k}_{D} {r}_{1}}}{{r}_{1}} - \frac{{e}^{- {k}_{D} {r}_{2}}}{{r}_{2}} ) ,$$

wherein: g₁ is an intensity autocorrelation function; ρ represents a distance between a light source and it light detector; τ represents delay time; μ′ₛ is a reduced scattering coefficient; k_{D} is a loss term related to photon absorption, scattering, and dynamic loss related to mean-square-displacement of scattering particles r₁=\sqrt{ρ²+(z−z₀)²}; r₂=\sqrt{ρ²+(z+z₀+2z_{b})²}; k_{D}=\sqrt{3 μₐμ′ₛ²+6 μ′ₛ²k₀²Γτ}; Γ=αD_{B}; D_{B} is a red blood cell diffusion coefficient; α is proportional to a volume of red blood cells in the local region; and k₀ is a photon wave number 2π/λ.

Another aspect of the invention provides a system including a diffuse correlation spectroscopy device and a pressure cuff.

This aspect of the invention can have a variety of embodiments. The system can further include a controller programmed to control operation of the diffuse correlation spectroscopy device and the pressure cuff in order to conduct diffuse correlation spectroscopy on a local region of the subject; apply pressure to restrict blood flow to the local region for a period of time; conduct diffuse correlation spectroscopy on the local region while the pressure is applied; release the pressure; and conduct diffuse correlation spectroscopy on the local region after the pressure is released.

The system can further include a diffuse near-infrared spectroscopy device.

### BRIEF DESCRIPTION OF THE DRAWINGS

For a fuller understanding of the nature and desired objects of the present invention, reference is made to the following detailed description taken in conjunction with the figures wherein:

FIG. 1 depicts a DCS system in accordance with embodiments of the invention;

FIG. 2 depicts autocorrelation plots in accordance with embodiments of the invention;

FIG. 3 depicts a cross-sectional model of a silicone flow phantom including small (<3 mm inner diameter) dear rubber tubing in a coil shape within a silicone phantom used to test embodiments of the invention;

FIG. 4 depicts a plot of diffusion coefficients against the known flow rates to determine the linearity of embodiments of the invention;

FIG. 5 depicts an integrated DCS/DNIRS probe in accordance with embodiments of the invention;

FIG. 6 depicts a method 600 of assessing peripheral arterial function in accordance with embodiments of the invention;

FIG. 7 depicts a system 700 for assessing peripheral arterial function in accordance with an embodiment of the invention;

FIG. 8 is at plot of blood flow indices (BFI) over time dining compression and following release as measured by embodiments of the invention;

FIG. 9 depicts experimental autocorrelation curves during baseline, during compression, and following release produced in accordance with embodiments of the invention;

FIG. 10 depicts a 3D plot of BFI obtained following changes in absorption and scattering coefficients in accordance with embodiments of the invention;

FIG. 11 depicts an example of collected BFIs over time during a compression protocol in accordance with embodiments of the invention;

FIG. 12 provides a chart of time-delay-to-reperfusion spike based upon peripheral artery disease severity as measured in accordance with embodiments of the invention (bars represent standard error, *p<0.02);

FIG. 13A depicts a capillary reperfusion spike relative to baseline values based on tobacco use as measured in accordance with embodiments of the invention (p<0.02)

FIG. 13B depicts capillary oxygen saturation based on tobacco use as measured in accordance with embodiments of the invention (p<0.02); and

FIG. 14 depicts hemoglobin concentrations of smokers and non-smokers as measured in accordance with embodiments of the invention.

### DEFINITIONS

The instant invention is most clearly understood with reference to the following definitions.

As used in the specification and claims, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

Unless specifically stated or obvious from context, as used herein, the term “or” is understood to be inclusive.

### DESCRIPTION OF THE INVENTION

One aspect of the invention utilizes Diffuse Correlation Spectroscopy (DCS) and optionally Diffuse Near Infrared Spectroscopy (DNIRS) to assess microcirculation in a subject. Embodiments of the invention are particularly useful for detecting and/or assessing the severity of Peripheral Arterial Disease (PAD) and/or vascular effects of smoking in adults.

The current diagnostic testing for Peripheral Arterial Disease, which uses Doppler ultrasound to assess the flow of blood through arteries, involves ultrasound examination and requires trained professionals to read the data, but also use subjective assessments of pulse volume recordings and patient history to ultimately diagnose the disease and its severity. Therefore, there is a clinical need for a quantitative assessment of arterial health that is independent of, and actually provides additional information towards, the patient's medical history (specifically smoking).

Diffuse Correlation Spectroscopy is a tool for assessing microvascular flow in deep (up to several centimeters) tissues. DCS utilizes the fluctuations in temporal intensity of multiply-scattered light to noninvasively quantify the movement of scatterers in the tissue (predominantly red blood cells). DCS has been validated against other modalities (namely arterial spin labeled MRI and Doppler Ultrasound). Similarly, DNIRS noninvasively assesses concentrations of oxy- and deoxy-hemoglobin, the two primary absorbers in the near infrared window (typically 650 nm to 850 nm).

### Optical Technologies

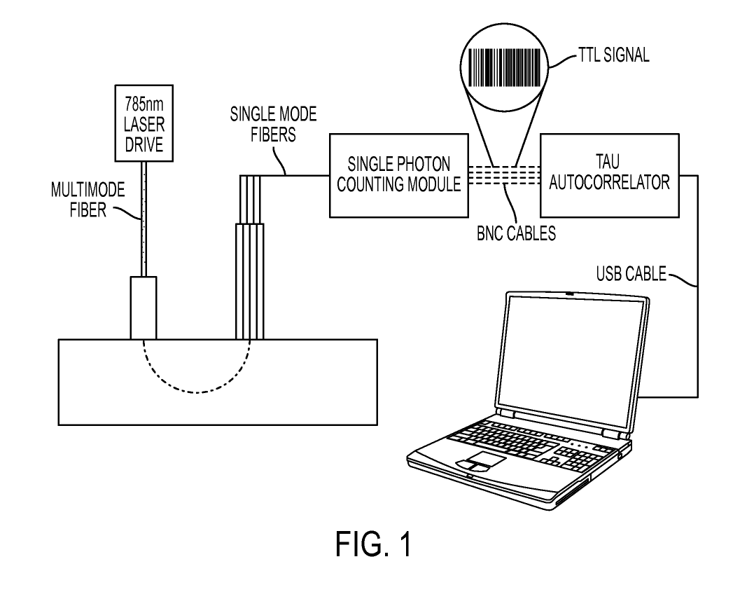

Diffuse Correlation Spectroscopy is an optical technology based upon the principles of photon correlation spectroscopy, which analyzes the temporal fluctuations of speckle light intensity due to interactions with moving particles, namely red blood cells. Dynamic light scattering or photon correlation spectroscopy is a method that has been used to study the dynamics of small (<10 μm) particle motions in solutions, biopolymers, and liquid crystals among others. FIG. 1 depicts a DCS system utilizing a 785 nm, long-coherence (˜10 m) laser, which maintains the phase of the light for the experimental path length (needed for analysis).

The optical fiber that delivers the light to the tissue is a multimode optical fiber, while the optical fiber which detects the light (e.g., 1.1 cm away) and connects to the detector (the next component to be described) is a single mode fiber (core diameter ˜5 μm), resulting in a penetration depth of approximately 5 mm into the tissue. A single-mode fiber is preferable as it can detect intensity fluctuations in a single speckle area. The detector for the DCS system can be a single photon counting module (SPCM) (Pacer, Palm Beach Gardens, Fla.). Each time a photon is detected, a 30 ns wide transistor-transistor logic (TTL) pulse (minimum of 2.5 volts) is outputted via a BNC connection.

The output of the SPCM is connected to a multi-tau autocorrelator (Correlator.com, Shen Zhen, China). The autocorrelator is arguably the center of the DCS system, converting the photon arrival times into the temporal correlation function of light scattered intensity used to calculate the diffusion coefficient. The multi-tau autocorrelator, separated into bins of sizes from 16 ns to several minutes, is designed to allow measurement times ranging from nanoseconds to hours, giving the system a dynamic temporal resolution while reducing the computational load of other autocorrelation systems. A complete description of how the autocorrelator operates can be found in C. Zhou, In-vivo optical imaging and spectroscopy of cerebral hemodynamics (2007) (Dissertation). In the unnormalized intensity autocorrelation function, the number of photons arrived in the i^{th} bin is multiplied by the photons from the 0^{th} (first) bin, as shown in equation (1)

G2(τᵢ)=<nᵢ·n₀> (1)

where < > is used to denote averaging, which is performed throughout the entire acquisition time. The overall autocorrelation function, G2(τ), is constantly updated and normalized before being passed to the computer.

The DCS system was validated using both single and multiple scattering regime techniques. Particles of known sizes ranging from 200-500 nm were measured in single scattering mode (7.5 parts per million). The DCS calculated size was determined using the calculated diffusion coefficient, D_{B}, according to Equation (2).

$${D}_{B} = \frac{{k}_{B} T}{6 πη r} \qquad ( 2 )$$

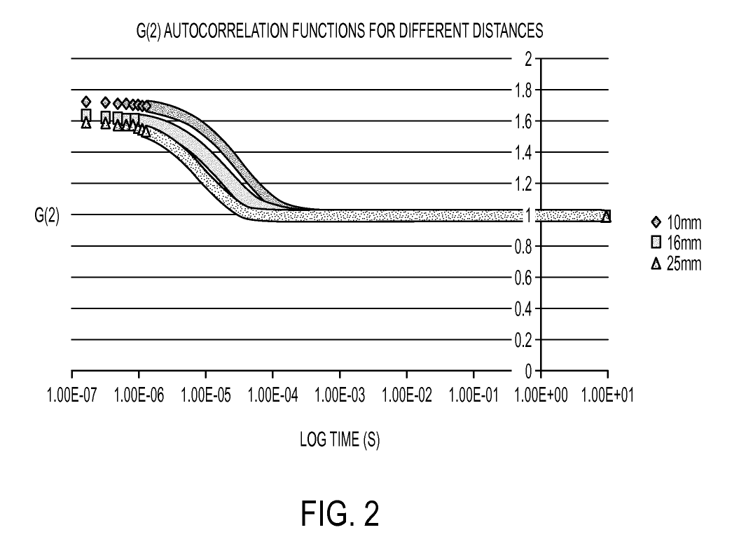

The computed sizes were statistically similar (p>0.05) to the sizes calculated by a commercial particle sizer (Malvern, Westborough, Mass.), which operates off the same principle of dynamic light scattering. In a multiple scattering regime, diffusion coefficients were calculated with source detector separations ranging from 10-25 mm in a beaker of 1% INTRALIPID® fat emulsion used in optical testing as it has the absorptive properties of water and the scattering properties of human tissue. In this test, it was expected that all three distances would yield the same diffusion coefficient, but greater source-detector separations would manifest as shifts in the autocorrelation function to the left, representative of the increased number of scattering events experienced during the path length of the photon compared to shorter separations. The autocorrelation function plots can be seen in FIG. 2, and the diffusion coefficients calculated had less than 4% standard deviation (7.9 E-9 cm²/s to 8.5 E-9 cm²/s),

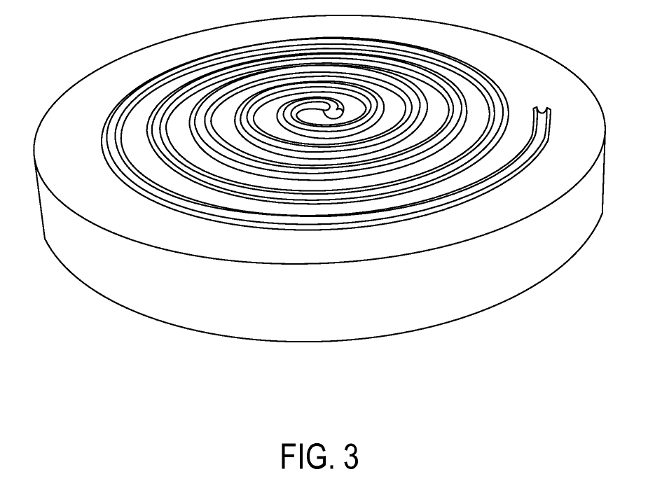

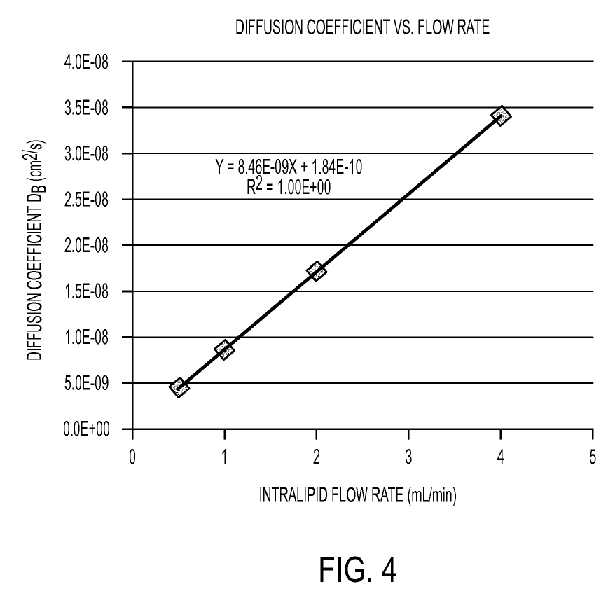

Finally, a flow phantom was created by inserting a small (<3 mm inner diameter) clear rubber tubing in a coil shape within a silicone phantom (modeled in FIG. 3). A controlled flow in 0.4% INTRALIPID® fat emulsion passed through the tubing at speeds ranging from 0.5-4 mL/min. The diffusion coefficients were plotted against the known flow rates to determine the linearity of the system, resulting in an r² of 1.00 as seen in FIG. 4.

For in-vivo work, the effective tissue blood flow can be characterized by using the red blood cell diffusion coefficient D_{B} and a parameter Γ=αD_{B}, where α is proportional to the volume of red blood cells in the tissue. The expression for the intensity autocorrelation function g₁ as an exponential function is provided in Equation (3) depending on the exponent, k_{D} and the terms r₁ and r₂, related to the root mean squared displacement of the light, and k₀, the photon wave number (2π/λ).

$$\begin{matrix}{g}_{1} ( ρ , τ ) = \frac{3 {μ}_{s}^{′}}{4 π} ( \frac{{e}^{- {k}_{D} {r}_{1}}}{{r}_{1}} - \frac{{e}^{- {k}_{D} {r}_{2}}}{{r}_{2}} ) & ( 3 ) \\ {r}_{1} = \sqrt{{ρ}^{2} + {( z - {z}_{0} )}^{2}} , {r}_{2} = \sqrt{{ρ}^{2} + {( z + {z}_{0} + 2 {z}_{b} )}^{2}} , \\ {k}_{D} = \sqrt{3 {μ}_{a} {μ}_{s}^{′2} + 6 {μ}_{s}^{′2} {k}_{0}^{2} Γτ} & ( 4 )\end{matrix}$$

It should be noted that the solutions listed above solve for the G₁ autocorrelation curve. The G₁ autocorrelation curve is based on fields, which cannot easily be directly measured. However, the G₂ autocorrelation function, which is based on right intensity, can be measured. Since the scattered field is Gaussian, the Siegert relation (G₂(r, τ)=1+β/g1(r, τ)/²) can be used to convert G₂ to G₁ and enable data fitting and eventual blood flow calculation. An example of a G₂ autocorrelation function can be seen in FIG. 2.

The G₂ solution requires the knowledge of the tissue's optical scattering and absorption coefficients. A Diffuse Near Infrared Spectroscopy (DNIRS) device can measure these coefficients and is described in M. S. Weingarten et al., “Diffuse near-infrared spectroscopy prediction of healing in diabetic foot ulcers: A human study and east analysis,” Wound Repair and Regeneration (2012) E. Papazoglou et al., “Assessment of diabetic foot ulcers with diffuse near infrared methodology” (2008); E. Papazoglou et al., “Noninvasive assessment of diabetic foot ulcers with diffuse photon density wave methodology: pilot human study,” 14 J. Biomedical Optics 064032 (2009); M. S. Weingarten, et al., “Prediction of wound healing in human diabetic foot ulcers by diffuse near infrared spectroscopy: A pilot study,” 18(2) Wound Repair and Regeneration 180-85 (2010).

Briefly, the DNIRS system includes six source fibers which deliver 70 MHz intensity-modulated light from laser-diodes at 685 nm or 830 nm wavelengths. The light passes through a MEMs optical switch that allows the transmission of one wavelength of light at a time through one source fiber. The light is then sent through the subsequent source fibers before the wavelength is changed and the process repeats for all source fibers and wavelengths. The backscattered light then is collected by two detector fibers located in the experimental probe between 4-16 mm from the various source fibers and is registered by avalanche photodiodes. The device then uses a quadrature demodulator to measure shifts in phase and changing amplitude in the scattered light compared to incident light, both as a function of source-detector separations.

This data was then fit into the diffusion approximation model and the optical absorption (μa) and reduced optical scattering (μs′) coefficients were calculated. The optical scattering and absorption coefficients can be used to then calculate the oxygen saturation and hemoglobin concentrations of the capillary beds in the measured tissue.

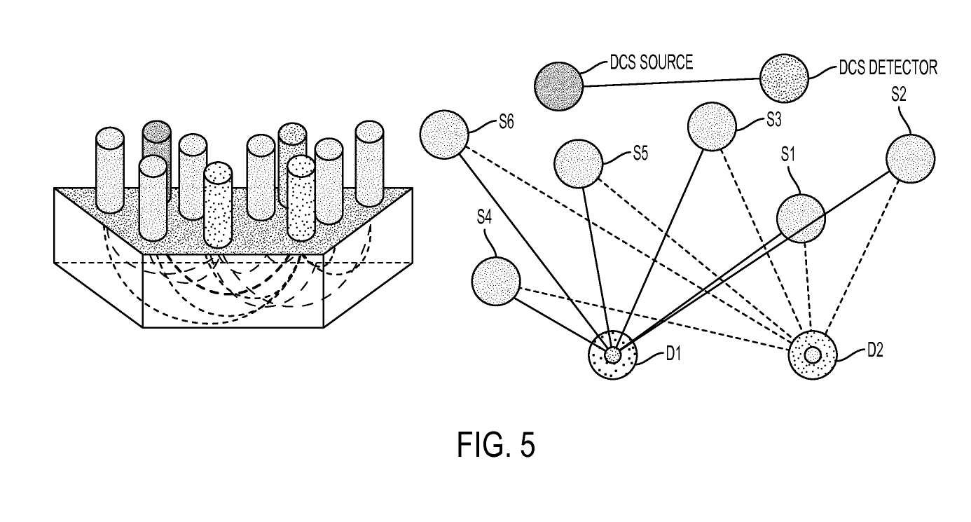

The DCS and DNIRS systems can be integrated such that all optical fibers connect to a single (PTFE) TEFLON® probe, shown in FIG. 5. It should be noted that the DNIRS system does not include a 785 nm wavelength laser, so the optical absorption and scattering coefficients are estimated using the 685 nm and 830 nm coefficients calculated by the DNIRS.

### Methods of Assessing Peripheral Arterial Function

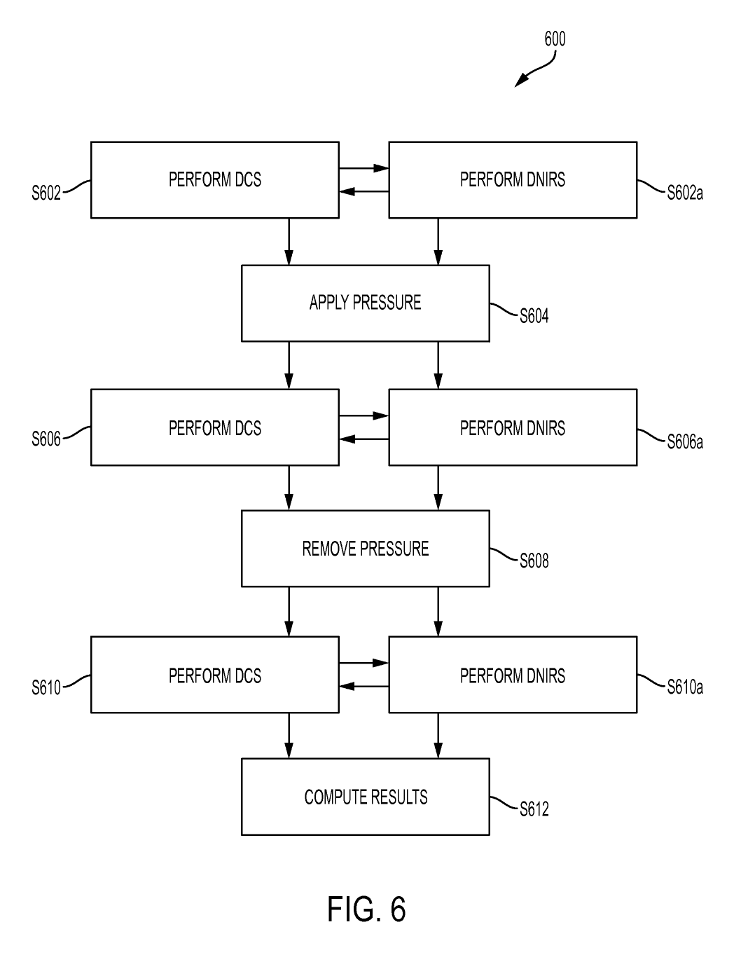

Referring now to FIG. 6, a method 600 of assessing peripheral arterial function is provided. This subject can be any animal, such as a human.

In step S602, diffuse correlation spectroscopy is performed on a local region of the subject. In one preferred embodiment, the local region is the ball of the subject's foot.

In step S602a, DNIRS is optionally performed to obtain the tissue's optical scattering and absorption coefficients. The optional DNIRS steps can be performed in between DCS measurements (e.g., about every 4 seconds, about every 8 seconds, and the like).

In step S604, pressure is applied to restrict blood flow to the local region for period of time. For example, a blood pressure cuff can be applied to the subject's calf and inflated to a pressure greater than the subject's systolic blood pressure (e.g., about 25 mm Hg greater than the subject's blood pressure). The period of time be can be, for example, between about 1 minute and about 2 minutes, between about 2 minutes and about 3 minutes, between about 4 minutes and about 5 minutes, or greater than about 5 minutes.

In step S606, DCS is performed on the local region while the pressure is applied and blood flow to the local region is restricted.

In step S608, the pressure is released.

In step S610, DCS is performed on the local region after the pressure is released.

In step S612, one or more results are calculated. These results can include: the magnitude of a spike between blood flow while the pressure is applied and blood flow after the pressure is released, a duration between release of the pressure and a peak of the spike, and a duration between release of the pressure and a return of blood flow to a pre-pressure level.

### Systems of Assessing Peripheral Arterial Function

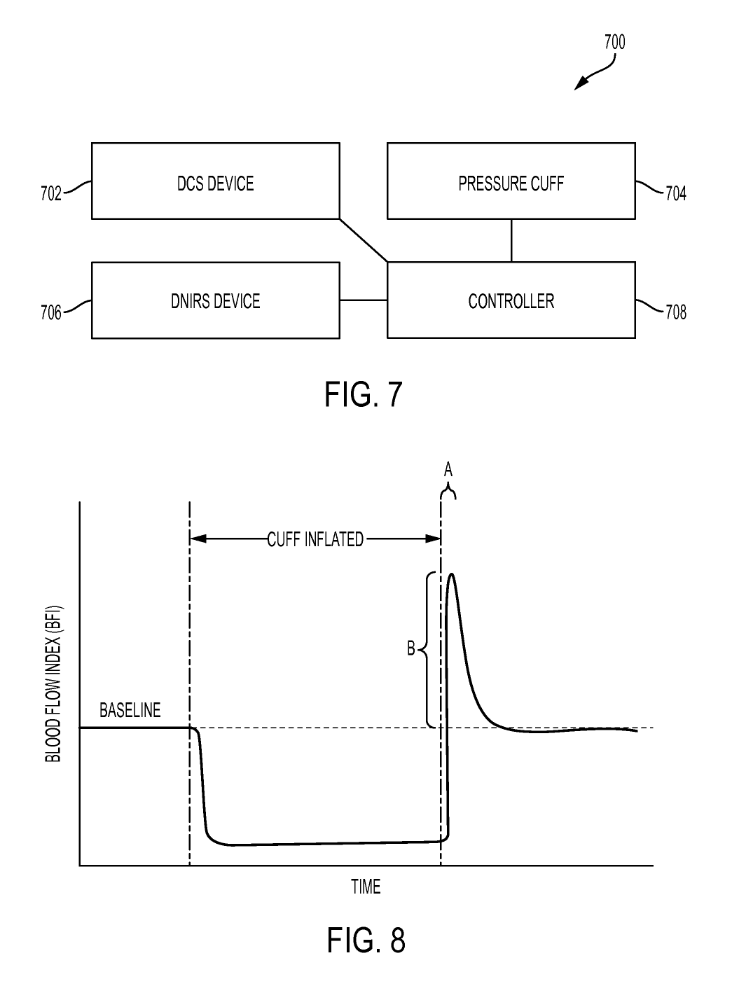

Referring now to FIG. 7, a system 700 for assessing peripheral arterial function is provided. System 700 can include a DCS device 702 and a blood pressure cuff 704. DCS device 702 can optionally also perform DNIRS as described herein. Alternatively, a separate DNIRS device 706 can be provided. A controller 708 (e.g., a general purpose computer programmed with appropriate software or a specially configured hardware device can communicated with DCS device 702 and/or DNIRS device 706 to obtain appropriate measurements and perform one or more the calculations discussed herein. Additionally or alternatively, controller 708 can communicate with and/or control operation of blood pressure cuff. Such an embodiment would enable a completely automated device.

### Working Example—Human Study

Twelve patients who were prescribed a segmental arterial study with pulse volume recording were recruited from the Drexel University Department of Surgery Vascular Laboratory. The study protocol was reviewed and approved by the Drexel University College of Medicine Institutional Review Board. Eligible patients were between the ages of 18 and 80 and had no known acute deep vein thromboses. Patients underwent, and completed, a routine segmental study, administered by a skilled ultrasound technologist and as prescribed by the treating physician. For the optical study, one blood pressure cuff was placed on the calf of one of the patient's symptomatic legs. This location was chosen as it was far enough away from the optical probe to not cause motion artifacts, yet was not too far up the leg to cause.

The optical probe was then placed on the ball of the foot and secured with silk medical tape. Baseline blood flow measurements were taken for 2 minutes (˜4 second temporal resolution), then the remaining cuff was inflated to 25 mm Hg higher than the ultrasound-determined systolic pressure. The cuff remained inflated for ˜4 minutes while continuous optical measurements were taken after which the pressure was released and the blood flow was monitored for an additional 2 minutes. At each individual measurement time point, the DCS device collected a single blood flow index with an averaging time of 1.5 seconds. Following this, the DNIRS device completed a single measurement (involving the scanning 4 separate wavelengths through 6 optical fibers). The DNIRS measurement took ˜2.5 seconds.

The blood flow index (BFI) at each time point was calculated and specific markers of disease were quantified, including the delay before a reperfusion spike occurred (interval A in FIG. 6) and the magnitude of the highest post-release flow recorded (relative to the patient's baseline now, interval B in FIG. 6). Data values were collected using a lab-designed LABVIEW® (National Instruments, Austin, Tex.) software interface and analyzed using MATLAB® (Math Works Inc., Natick, Mass.) software. The patient diagnoses, and brief vascular medical history (including tobacco use, diabetes status, and use of high blood pressure or cholesterol medications), were gathered and then compared to the calculated optical values and time points mentioned above. Student's T-tests were used as they are the typical statistical test for determining differences between two groups.

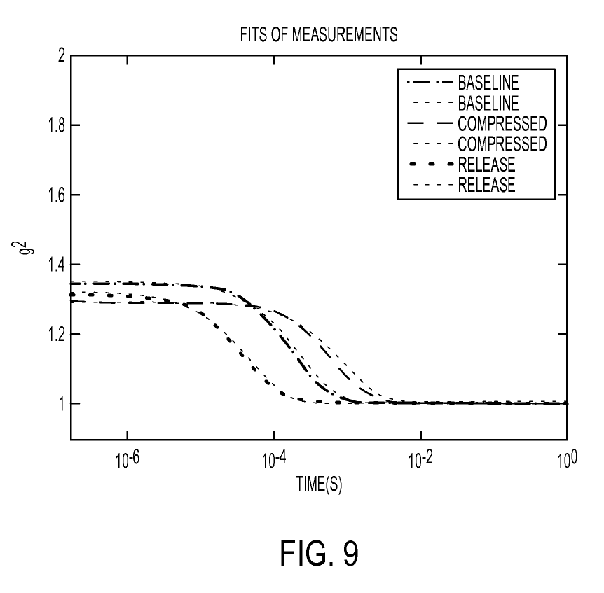

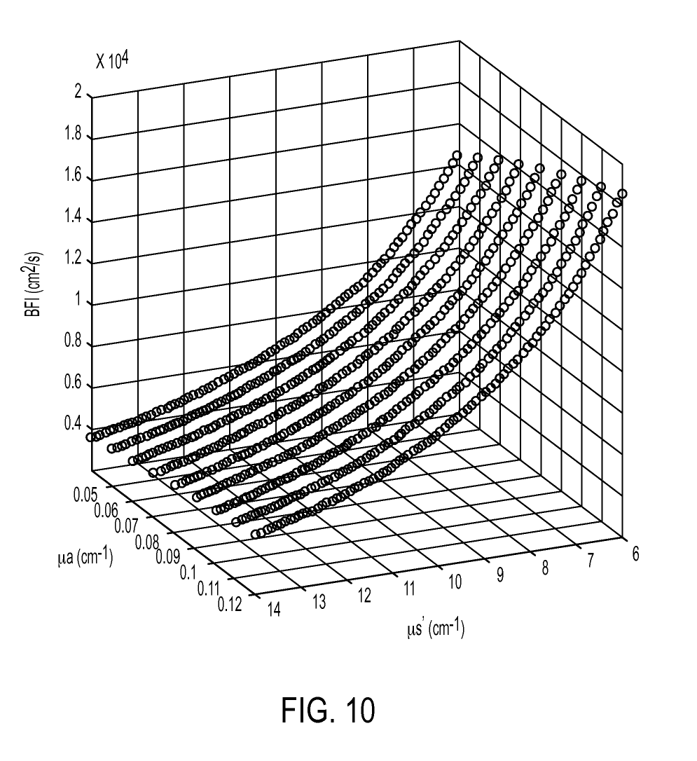

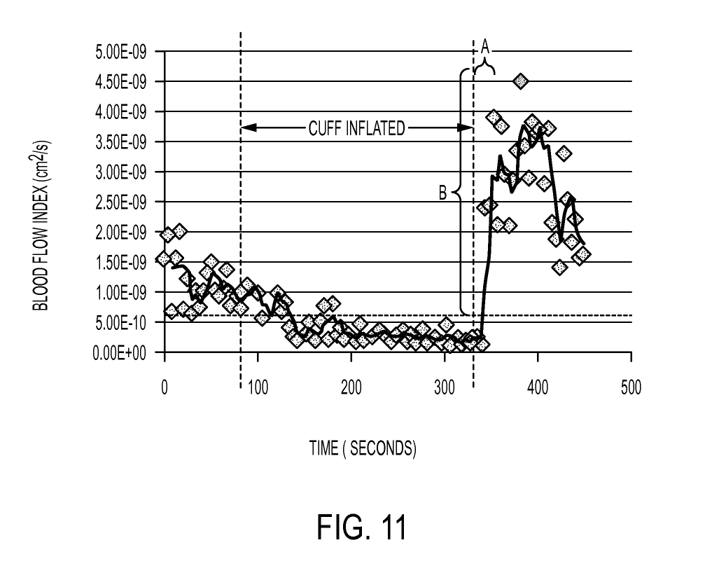

A typical experimental autocorrelation curve is shown in FIG. 9. The data show that when the blood flow is compressed, the autocorrelation curve shifts to the right (representing longer time delays and consequently slower blood flow), and following release, where blood flow is fastest (see maximum value of FIG. 8), the curve shifts to the left and exhibits a steeper exponential decay compared to baseline, as expected. As the DCS solution is predominantly dependent upon the scattering coefficient (see Equation (3)), a small error in the μs′ can lead to large changes in the BFI, as seen in FIG. 10. In this figure, BFIs were calculated using Equation 3 and it can be seen that doubling the absorption minimally affects (<10%) the BFI, whereas doubling the scattering changes the BFI by nearly 400%. As the DNIRS and DCS systems alternated, marginal variations in the tissue conditions from one second to the next and resulted as errors in the optical coefficients. These errors, combined with motion artifacts, manifested as the noticeable noise during the experiment, seen in FIG. 11.

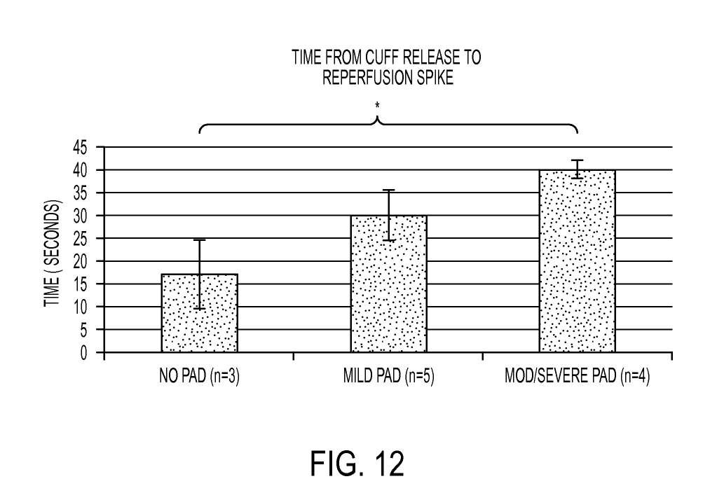

Of the 12 patients enrolled in the study, three were diagnosed as having no PAD, five had mild PAD, two had moderate PAD, and two had severe PAD. It was hypothesized that PAD severity would link to the delay in reperfusion (represented as “A” in FIG. 6) following cuff release. As shown in FIG. 12, this trend was evident, with statistical difference (p<0.02) between the patients with moderate/severe PAD and healthy patients. It is expected that this trend would prove to be statistically significant between all 3 groups with a larger sample size. It seems logical that PAD severity would cause the impairment in the process to deliver oxygenated blood to tissue with a sudden high oxygen demand. This would correlate with intermittent claudication, the pain experienced by patients with PAD during walking or mild exercise.

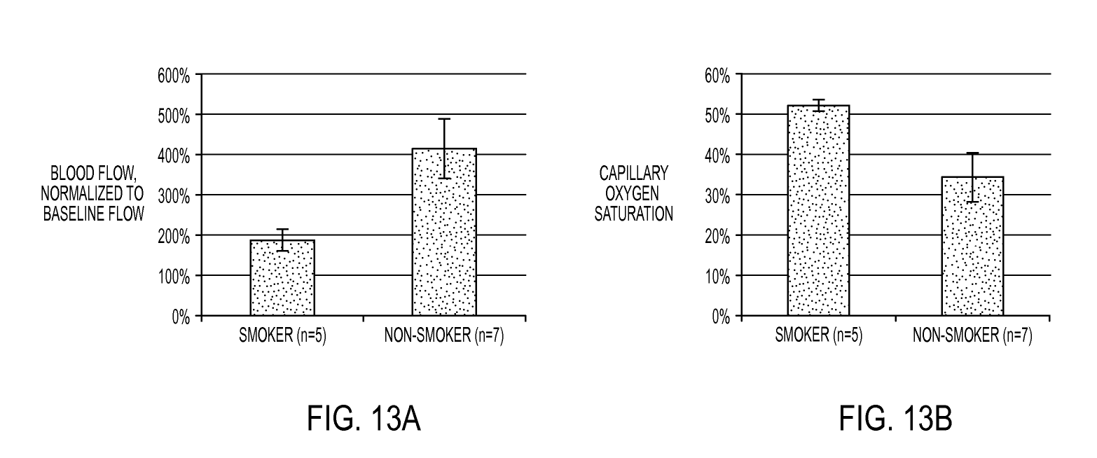

In assessing the medical history of the patients, it was discovered that cigarette smoking was associated with the phenomenon of an impaired reperfusion spike. A comparison of smoking and non-smoking reperfusion spikes (indicated as “B” in FIG. 8), as a relative percent of the patient's baseline blood flow, is shown in FIG. 13A. It can be seen that the average non-smoking patient had a reperfusion spike of over 400% of their baseline flow, whereas smokers had a spike of less than 200% (p<0.02). Of the non-smokers, those who were former smokers (n=3) averaged a spike of 290%, compared to 510% for those who never smoked (n=4) showing a correlation between tobacco use and an impaired reperfusion spike. The data show that tobacco use results in reduced arterial compliance, limiting the ability of the vessels to dilate when there is a sudden oxygen demand.

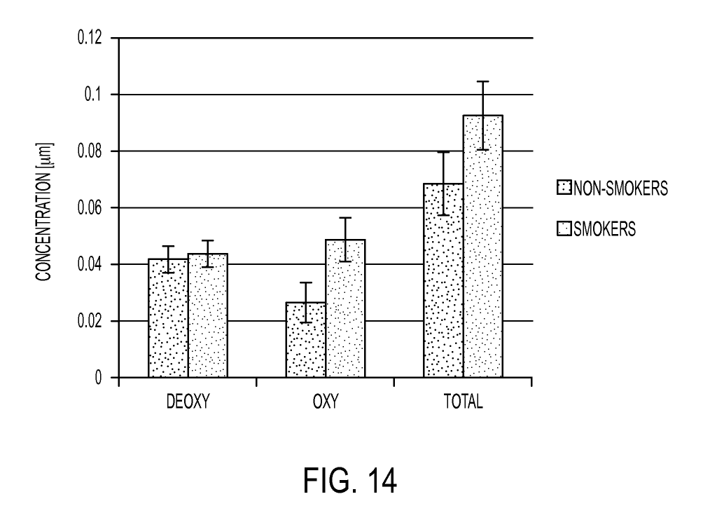

A second interesting optical difference between smokers and non-smokers involved the oxygen saturation, as assessed with the DNIRS optical system. As seen in FIG. 13B, smokers had a statistically (p<0.02) higher oxygen saturation than non-smokers. It was further determined that smokers and non-smokers had similar quantities of deoxygenated hemoglobin, but smokers had nearly twice as much oxygenated hemoglobin (shown in FIG. 14). These results indicate that smokers have impairment in the process of delivery oxygen to the tissue in the capillaries. This is corroborated by previous reports from literature which show that smoking causes an increase in red blood cell count and results in blood cells with a higher oxygen affinity (impairing the ability to release oxygen when needed). It is worthy of notice that carboxyhemoglobin (hemoglobin with bound carbon monoxide and as known biological product of smoking) does not absorb light at the same wavelength as oxygenated hemoglobin (absorption at 785 nm is an order of magnitude lower for carboxyhemoglobin than oxygenated hemoglobin), ensuring that the chromophore being assessed was indeed oxy-hemoglobin.

Several hemodynamic abnormalities were documented using embodiments of the invention. First, endothelial health was assessed by monitoring reperfusion rates and magnitudes. Previous studies have found that peripheral arterial disease is associated with impaired flow mediated dilitation, resulting in delayed reperfusion. The delayed reperfusion spike seen in patients with PAD also matches the results presented using similar optical techniques, albeit with a single patient in the PAD group. Studies have also shown that smoking can cause an impaired reperfusion, as assessed using ultrasound to measure arterial diameter following compression.

The results herein validate the use of a novel technique (flow-mediated dilatation assessment by Diffuse Correlation and Near Infrared Spectroscopies) to study arterial compliance in patients with impaired endothelial function, specifically relating to peripheral arterial disease severity and tobacco use. Aspects of the invention can provide supplemental information which may have been overlooked using subjective methodologies or provide a rapid screening technique which can be performed by non-medical experts.

### EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

The entire contents of all patents, published patent applications, and other references cited herein are hereby expressly incorporated herein in their entireties by reference.

1. A method for assessing peripheral arterial function in a subject, the method comprising:
conducting diffuse correlation spectroscopy on a local region of the subject;
applying pressure to restrict blood flow to the local region for a period of time;
conducting diffuse correlation spectroscopy on the local region while the pressure is applied;
releasing the pressure; and
conducting diffuse correlation spectroscopy on the local region after the pressure is released.

2. The method of claim 1, wherein the local region is a ball of the subject's foot.

3. The method of claim 1, wherein the pressure is applied by a blood pressure cuff.

4. The method of claim 1, wherein the pressure is equal to or greater than the subject's systolic blood pressure.

5. The method of claim 1, wherein the pressure is about 25 mm Hg greater than the subject's systolic blood pressure.

6. The method of claim 1, further comprising:
calculating a spike between blood flow while the pressure is applied and blood flow after the pressure is released.

7. The method of claim 6, further comprising:
calculating a duration between release of the pressure and a peak of the spike.

8. The method of claim 1, further comprising:
calculating a duration between release of the pressure and a return of blood flow to a pre-pressure level.

9. The method of claim 1, wherein the period of time is selected from the group consisting of: between about 1 minute and about 2 minutes, between about 2 minutes and about 3 minutes, between about 4 minutes and about 5 minutes, and greater than about 5 minutes.

10. The method of claim 1, wherein the steps of conducting diffuse correlation spectroscopy comprise:
applying light to a first location of the subject's skin;
detecting photons resulting from interactions between the light and moving objects under the subject's skin;
correlating arrival times of the photons with light scattered intensity; and
calculating a diffusion coefficient based on autocorrelation of the light scattered intensity.

11. The method of claim 10, wherein the light applied to the subject's skin is near-infrared light.

12. The method of claim 11, wherein the light applied to the subject's skin has a wavelength between about 650 nm and about 1,000 nm.

13. The method of claim 12, wherein the light applied to the subject's skin has a wavelength of about 785 nm.

14. The method of claim 10, wherein the light is generated by a long-coherence laser.

15.-18. (canceled)

19. The method of claim 10, wherein the correlating step utilizes a multi-tau autocorrelation algorithm.

20. (canceled)

21. (canceled)

22. The method of claim 10, wherein the steps of conducting diffuse correlation spectroscopy further comprise:
generating a transistor-transistor logic (TTL) pulse each time a photon is detected.

23. The method of claim 10, wherein the steps of conducting diffuse correlation spectroscopy further comprise:
performing diffuse near-infrared spectroscopy (DNIRS) to determine the skin's optical scattering and absorption coefficients.

24. A method for assessing peripheral arterial function in a subject, the method comprising:
conducting diffuse correlation spectroscopy on a local region of the subject;
applying pressure to restrict blood flow to the local region for a period of time;
conducting diffuse correlation spectroscopy on the local region while the pressure is applied;
releasing the pressure; and
conducting diffuse correlation spectroscopy on the local region after the pressure is released;
wherein the steps of conducting diffuse correlation spectroscopy comprise:
applying light to a first location of the subject's skin;
detecting photons resulting from interactions between the light and moving objects under the subject's skin;
correlating arrival times of the photons with light scattered intensity;
calculating a diffusion coefficient based on autocorrelation of the light scattered intensity; and
solving the equation
$${g}_{1} ( ρ , τ ) = \frac{3 {μ}_{s}^{′}}{4 π} ( \frac{{e}^{- {k}_{D} {r}_{1}}}{{r}_{1}} - \frac{{e}^{- {k}_{D} {r}_{2}}}{{r}_{2}} ) ,$$
wherein:
g₁ is an intensity autocorrelation function;
ρ represents a distance between a light source and a light detector;
τ represents delay time;
μ′ₛ is a reduced scattering coefficient;
k_{D} is a loss term related to photon absorption, scattering, and dynamic loss related to mean-square-displacement of scattering particles;
r₁=\sqrt{ρ²+(z−z₀)²};
r₂=\sqrt{ρ²+(z+z₀+2z_{b})²};
k_{D}=\sqrt{3 μ_{α}μ′ₛ+6μ′ₛ²k₀²Γτ};
Γ=αD_{B};
D_{B} is a red blood cell diffusion coefficient;
α is proportional to a volume of red blood cells in the local region; and
k₀ is a photon wave number 2π/λ.

25. A system comprising:
a diffuse correlation spectroscopy device;
a pressure cuff; and
a controller programmed to control operation of the diffuse correlation spectroscopy device and the pressure cuff in order to:
conduct diffuse correlation spectroscopy on a local region of the subject
apply pressure to restrict blood flow to the local region for a period of time;
conduct diffuse correlation spectroscopy on the local region while the pressure is applied;
release the pressure; and
conduct diffuse correlation spectroscopy on the local region after the pressure is released:
wherein the diffuse correlation spectroscopy includes solving the equation g₁(ρ, τ)=3μ′ₛ/4π(e^{-k}^{_{D}}^{r}^{₁}/r₁−e^{-k}^{_{D}}^{r}^{₂}/r₂)g₁(ρ, τ)=3μ′ₛ/4π(e^{-k}^{_{D}}^{r}^{₁}/r₁−e^{-k}^{_{D}}^{r}^{₂}/r₂), g₁(ρ, τ)=3μ′ₛ/4π(e^{-k}^{_{D}}^{r}^{₁}/r₁−e^{-k}^{_{D}}^{r}^{₁}/r₁−e^{-k}^{_{D}}^{r}^{₂}/r₂) wherein:
g₁ is an intensity autocorrelation function;
ρ represents a distance between a light source and a light detector;
τ represents delay time;
μ′ₛ is a reduced scattering coefficient;
k_{D} is a loss term related to photon absorption, scattering, and dynamic loss related to mean-square-displacement of scattering particles;
r₁=\sqrt{ρ²+(z−z₀)²}:
r₂=\sqrt{ρ²+(z+z₀+2z_{b})²}:
k_{D}=\sqrt{3μ′ₛ²+6μ′ₛ²k₀²Γτ};
Γ=αD_{B}:
D_{B} is a red blood cell diffusion coefficient;
α is proportional to a volume of red blood cells in the local region; and
k_{D} is a photon wave number 2π/λ.

26. (canceled)

27. The system of claim 25, further comprising:
a diffuse near-infrared spectroscopy device.
